(19) United States
(12) Patent Application Publication
(10) Pub. No.: US 2004/0193040 A1
(43) Pub. Date: Sep. 30, 2004

(54) Method for imaging a metabolic event of an organism

(76) Inventors: Guentr Brill, Saarbruecken (DE); Michael Deimling, Moehrendorf (DE); Hermann Requardt, Erlangen (DE)

Correspondence Address: SCHIFF HARDIN, LLP, PATENT DEPARTMENT, 6600 SEARS TOWER, CHICAGO, IL, 60606-6473, US

(21) Appl. No.: 10/741,776

(22) Filed: Dec. 19, 2003

(30) Foreign Application Priority Data
Dec. 19, 2002 (DE) 10259793.6

(51) Int. Cl. A61B 5/055

(52) U.S. Cl. 600/420

(57) ABSTRACT

In a method for imaging a metabolic event of an organism, a substance (involved in the metabolism) to be imaged is marked with a substance that exhibits a high T1 and is polarized. The marked and polarized substance involved in the metabolism is administered to the organism. An image of a region of the organism is generated with a magnetic resonance device, this image showing the distribution of the polarized substance in the region.

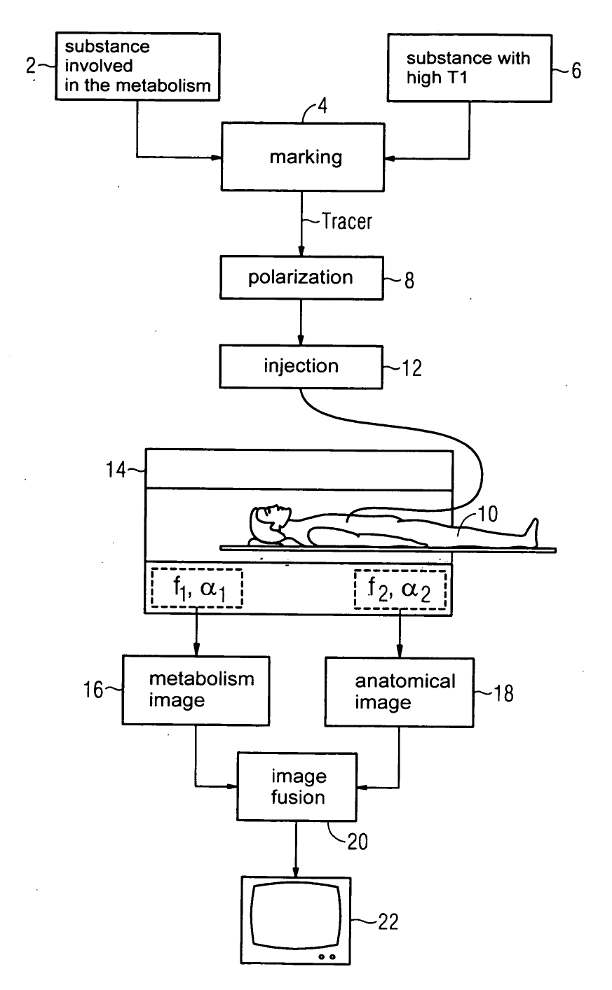

### BACKGROUND OF THE INVENTION

1. Field of the Invention

The present invention concerns a method for imaging a metabolic event of an organism.

2. Description of the Prior Art

Metabolic events of an organism can be graphically represented by means of positron emission tomography (PET). The particular properties of positron emitters and the positron annihilation are utilized in order to quantitatively determine the functioning of organs or cell regions. The measurement principle is to use tracers, which are marked with a positron emitter. The positron emitters used most in PET are ¹¹C, ¹³N, ¹⁵O and ¹⁸F. The replacement of a stable isotope in a biomolecule with positron emitters ¹¹C, ¹³N, and ¹⁵O causes no change in the biochemistry of the tracer, and thus enables the undisturbed imaging of their metabolic behavior. Changes in the metabolic behavior given the use of 18F, which frequently replaces hydrogen in biomolecules, are desired or so minimal that they do not cause substantial disturbance. Thus, for example, 18F-FDG is used as a tracer for measurement of the glucose metabolism, and, for example, F-DOPA is used for display of the dopamine metabolism. Clinical applications of PET are, among other things, cardiology, neurology and oncology. The simultaneous imaging of entire volume regions, in which the metabolism and the biochemistry can be quantitatively shown in vivo, has proven to be particularly advantageous. Due to the short half-life, however, the radioactive marker in use is produced on site, undergoes a quality control, and is then injected into the patient. Furthermore, the anatomical detailing, with 1 or 2 mm for specialized brain tomographs and 2 to 3 mm for whole-body tomograms, is insufficient in many cases. Modern systems therefore have an x-ray computed tomography device (CT device) downstream. The anatomic images generated with the CT device are fused in a post-processing step.

It is also possible by means of magnetic resonance technology to graphically show the concentration of, for example, ¹⁹fluorine in an organism. The low concentration of fluorine in the organism, and thus a low sensitivity to magnetic resonance technology, has a disadvantageous effect on fluorine imaging. Conventionally, this has been compensated by large voxels in the image data, thus a correspondingly lower spatial resolution.

A method for magnetic resonance imaging is described in U.S. Pat. No. 6,278,893, in which a contrast agent is used that has a high T1 relaxation time and that is polarized ex vivo. Such contrast agents are formed of nuclei with a non-zero magnetic moment. For example, ¹⁹F, ³Li, ¹H, ¹³C, ¹⁵N or ³¹P are suitable. The acquired contrast agent images are then superimposed on anatomical images, i.e. proton images. It is also described in this patent that, by means of adapted radio-frequency excitation, or by means of phase-sensitive methods, magnetic resonance images of nuclei that are present only in various chemical environments can be generated. For imaging, use is made of the fact that, in contrast agents with a high T1 relaxation time (that in particular are ¹⁹F nuclei and ¹³C nuclei), the chemical shift; changes dependent on a metabolic activity. This activity can be used for graphical representation.

### SUMMARY OF THE INVENTION

An object of the present invention is to provide a method for metabolic imaging with high spatial resolution, wherein no radioactive markers are used.

This object is achieved by polarizing a substance involved in the metabolism to be imaged that is marked with a substance that exhibits a high T1, administering the marked and polarized substance involved in the metabolism to the organism, and generating, with a magnetic resonance device, an image of a region of the organism, the image showing the distribution of the polarized substance in the region.

It is advantageous to be able to use basically the same substances that are also used in PET, after a corresponding marking by a nucleus with a magnetic moment. However, in comparison with PET, here a repetition of the measurement after a relatively short time is possible. This time is determined by the decay of the polarization. The method can be implemented with a suitably equipped diagnostic magnetic resonance device.

In an embodiment, as marked and polarized metabolic starting material, tracers are used that are also in principle used in PET, but the radioactive markers are replaced by non-radioactive markers that possess a nuclear-magnetic moment. This has a simplifying effect on the governmental approval procedure required in many countries for new medical uses of substances.

Given the use of ¹⁹F as a marker, with modern methods of polarization (hyperpolarization) the population distribution of the spin states of this marker can be easily increased from 10⁻⁶ to 0.2. Sufficient signal-emitting nuclei are available for this in order to achieve spatial resolutions in the range of millimeters, which are also achieved with PET.

Many metabolic events occur in a time range of minutes. In order to show such a metabolic event, and not only the vessel volume, a quasi-continuous administration of the marker ensues over a duration of minutes. Administration and polarization are then undertaken simultaneously.

In order to obtain information about the time curve of the accumulation or the perfusion of the polarized substance involved in the metabolism, images with lower resolution (for example 64×128) and/or very small flip angles of less than 1° can be generated intermittently with the normal imaging. The polarization curve is only marginally disturbed, and the signal-to-noise ratio is sufficiently good due to the small matrix size.

### DESCRIPTION OF THE DRAWINGS

The figure illustrates an exemplary embodiment of the invention.

### DESCRIPTION OF THE PREFERRED EMBODIMENTS

The inventive method for imaging a metabolic event of an organism is suited in particular for graphical display of the glucose metabolism and of the dopamine metabolism. Given the use of suitable tracers, however, it is also suitable for imaging of the fatty-acid metabolism and of the amino acid metabolism, or the perfusion. Clinical applications for the graphical display of the glucose metabolism are used in cardiology, neurology, and oncology. By the graphical display of the dopamine metabolism, most notably the dopamine pool can be determined, and from this conclusions can be made about the pre-synaptic dopamine function. The F-DOPA that is used serves as a neurotransmitter in the brain, and can be used with good effect in the early detection of Parkinson's disease and Alzheimer's disease.

The inventive method for imaging the metabolic begins with a metabolic participant 2 of the metabolism to be imaged, such as, for example, F-fluorine deoxyglucose (F-FDG) in the glucose metabolism and F-DOPA in the dopamine metabolism. A substance 6 with a high T1 is used to mark the substance 2 involved in the metabolism. In the example, for this the fluorine present in the substance 2 involved in the metabolism is replaced by the fluorine isotope ¹⁹F. Since the population inversion of ¹⁹F at body temperature and at approximately 1 Tesla is only 10⁻⁶, the marked substance 2 involved in the metabolism is polarized ex vivo before the application with a known method for hyperpolarization (method step 8). For example, the accumulation of para-hydrogen relative to ortho-hydrogen can be utilized at lower temperatures (T<20K). This polarization is transferred to a solid-state material via a catalytic application reaction on an organic substrate or a metal complex. The polarization is stored due to the very long T1 time in the solid body. Finally, the polarization is transferred to the fluorine nuclei by polarization transfer (cross-relaxation). The hyperpolarization also can ensue, for example, by optical pumps.

The thusly-polarized tracer is then administered to an organism 10 in the form of, for example, an intravenous solution, for imaging of the corresponding metabolic event (method step 12). This ensues quasi-continuously up to a plurality of minutes dependent on the metabolic event to be imaged. The polarization is then simultaneously effected. For actual (real) imaging of the metabolic event, a magnetic resonance device 14 is used that is fashioned for imaging of two different nuclei types. An excitation and a further processing of magnetic resonance signals ensues for the fluorine nuclei for metabolism imaging, and for protons for conventional imaging of the anatomy. The substantial difference is in the magnetic resonance frequencies of both nuclei whose distribution is graphically displayed. Given a basic magnetic field of 1 Tesla, the magnetic resonance frequency is approximately f₁=40 MHz for fluorine nuclei and f₂=42 MHz for proton imaging. The magnetic resonance device 14 therefore must be suitably fashioned only in its radio-frequency stage, including the radio-frequency antennas, and in the control of the gradient fields for spatial coding, and in the signal evaluation.

In particular, fast sequences, such as a 2D or 3D FLASH sequence, are suitable as imaging sequences, meaning a specific series of radio-frequency fields for excitation and of gradient fields for spatial coding. FLASH is an abbreviation for Fast Angle Low Shot, a rapid gradient echo sequence. In the imaging of hyperpolarized fluorine, it must be taken into account that the excitation angle α₁ (flip angle) for fluorine imaging is only in the range of approximately 1°, also smaller than 1° in the aforementioned imaging with lower resolution. Additionally or alternatively, the matrix size can be reduced. This is because in each excitation, a corresponding part of the polarization corresponding to

M_{z}(n)=M_{hyperpole}·cos(αₙ)·exp(−n·TR/T1)

with T1 relaxation time of the hyperpolarized nucleus

TR repetition time

N number of the radio-frequency excitations

αₙ flip angle

is needed. A high signal M_{z}(n)·sin(αₙ) is available due to the hyperpolarization of the imaging fluorine nuclei. By contrast, for proton imaging the excitation angle α₂ is selected corresponding to the desired image weighting.

The temporal control of the image exposure must be considered to be sure that the bolus of the marked and polarized substance involved in the metabolism has reached the region to be examined in the patient 10 at a specific time after the injection 12, and is effective in that region. Only then does the image exposure of a metabolism image 16 begin. Before or after the metabolism imaging, a conventional anatomical magnetic resonance image 18 is generated. From the metabolism image 16 and the anatomical image 18, a superimposed image 20 that can be shown on a display device 22 is generated by image fusion after suitable registration.

The method for imaging a metabolic event is not limited to marking with fluorine. Tracers also can be used that are marked with other isotopes that exhibit a high T1 in the molecular environment. For example, ¹³C, ¹⁵N, ³¹P or ³Li are suitable,

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for imaging a metabolic event of an organism, comprising the steps of:
marking a substance, as a marked substance, involved in a metabolism to be imaged by magnetic resonance, with a marking substance exhibiting a high T1, and polarizing the marked substance;
administering the marked and polarized substance involved in the metabolism to an organism; and
generating an image of a region of the organism, in which said metabolic event occurs, with a magnetic resonance imaging apparatus, said first image representing a distribution of said marked and polarized substance in said region.

2. A method as claimed in claim 1 wherein said image is a first image, and comprising generating a second image of said region with said magnetic resonance apparatus representing a distribution of protons in said region, and generating an overall image of said region by using said first image and said second image.

3. A method as claimed in claim 2 comprising selectively exciting said marked and polarized substance with respect to its Larmor frequency to generate said first image.

4. A method as claimed in claim 2 comprising generating said first image using a magnetic resonance imaging sequence in said magnetic resonance apparatus having an excitation pulse with a flip angle of approximately 1°.

5. A method as claimed in claim 2 wherein said protons have a Larmor frequency that is different from the Larmor frequency of the marked and polarized substance, and comprising selectively exciting said protons with regard to the Larmor frequency of said protons to generate said second image.

6. A method as claimed in claim 1 comprising using ¹⁹F as said marking substance.

7. A method as claimed in claim 1 comprising employing a material involved in glucose metabolism as said marked substance.

8. A method as claimed in claim 1 comprising employing ¹⁹F deoxyglucose as said marked substance.

9. A method as claimed in claim 1 comprising using F-DOPA as said marked substance.

10. A method as claimed in claim 1 comprising administering said marked and polarized substance to said organism quasi-continuously for a duration comprising a plurality of minutes.

11. A method as claimed in claim 1 comprising polarizing said marked substance simultaneously with administration thereof.

12. A method as claimed in claim 1 comprising generating a plurality of images of said region with said magnetic resonance apparatus.

13. A method as claimed in claim 12 comprising generating at least one of said plurality of images with a lower resolution than a remainder of said plurality of images.

14. A method as claimed in claim 12 comprising generating at least one of said plurality of images with a magnetic resonance imaging sequence having an excitation pulse with a flip angle that is smaller than a flip angle of an excitation pulse in a magnetic resonance imaging sequence used to generate a remainder of said plurality of images.
